(19) United States
(12) Patent Application Publication
(10) Pub. No.: US 2019/0167674 A1
(43) Pub. Date: Jun. 6, 2019

(54) VIGOR ENHANCEMENT VIA ADMINISTRATION OF PYRIMIDINE DERIVATIVES

(71) Applicant: University of Central Florida Research Foundation, Inc., Orlando, FL (US)

(72) Inventors: Kiminobu SUGAYA, Orlando, FL (US); Angel ALVAREZ, Birmingham, AL (US)

(21) Appl. No.: 16/269,243

(22) Filed: Feb. 6, 2019

Related U.S. Application Data
(63) Continuation of application No. 15/726,533, filed on Oct. 6, 2017, now abandoned.
Application No. 15/726,533 is a continuation of application No. 11/563,891, filed on Nov. 28, 2006, now abandoned.

(51) Int. Cl. A61K 31/497

(52) U.S. Cl. CPC A61K 31/497

(57) ABSTRACT

Disclosed herein are methods for increasing the overall vigor of a subject, and/or vigor of target tissues of a subject. Exemplified herein is the utilization of pyrimidine derivatives which act to stimulate stem cell proliferation. In addition to increasing vigor, such stem cell proliferation agents (SCPA) may be used to enhance and/or improve the outcome of other therapies, and may be used in psychiatric applications. Increasing vigor in subjects is not necessarily targeted to the treatment of a disease, thus, the methods can include administration to clinically healthy animals.

### BACKGROUND

Much research is being conducted to study stem cells and to devise ways of utilizing stem cells in treating various neurological pathologies and injuries, as well as pathologies of other organ systems. It is generally recognized that stem cell technologies hold tremendous promise for ultimately treating and even curing neurologically related diseases, injuries or dysfunctions. It is understandable why stem cell research has focused on these areas. However, finding new and more diverse ways of utilizing stem cells is an ongoing challenge. It is important that stem cell research be directed to beneficial areas that do not necessarily include the traditional areas of developing treatments and cures for disease and injuries. Furthermore, agents that affect the characteristics of stem cells should be studied as this may reveal certain useful compounds that can be utilized to manipulate endogenous stem cells and thus leading to novel therapies.

### DETAILED DESCRIPTION

The subject invention is based on the inventors' realization that relatively little research and development has been conducted in the area of stem cell-based applications for healthy individuals or subjects. Accordingly, in one embodiment, the subject invention pertains to a method of increasing vigor of a human or nonhuman subject that comprises the administration of a vigor-enhancing composition that contains stem cell proliferating agent (SCPA). U.S. Pat. Nos. 5,976,523 ('523 patent) and 4,959,368 ('368 patent) teach a number of compounds that may be used as wound healing agents. The '523 patent teaches that the wound healing agents described therein act by potentiating growth factors and cytokines released in tissues as a result of injury or wounding of tissues. Essentially, the '523 patent teaches that the agents stimulate the migration of cells toward the wound. The present inventors have discovered that the same agents actually stimulate the proliferation of stem cells, which in turn, led to the discovery that the agents may be used in circumstances where tissues have not been wounded.

Accordingly, the agents presented in the '523 patent and '368 patent are incorporated herein by reference for disclosure of SCPA agents. Formulas 1 and 2 as set forth in the '523 patent are provided:

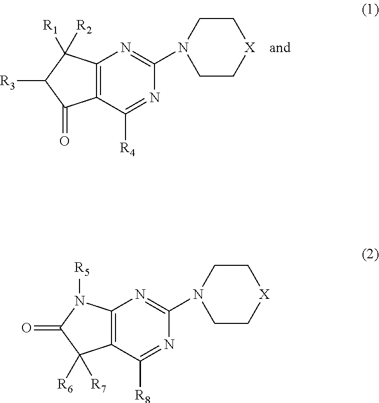

wherein R₁ to R₈ independently represent a hydrogen atom, a lower alkyl (especially C₁-C₇ alkyl) group, CH₃OCH₂CH₂—, —CH₂CONH₂, —COCH₃, —COC₂H₅ or —CH₂OCOC₂H₅, and X represents ═NH, ═N—CH₃, ═N—COCH₃, ═N—COOC₂H₅, ═N—SO₂CH₃, ═CH₂, ═CHCH₃, ═CHC₂H₅, —O— or —S— in which ph stands for a phenyl group.

Typical illustrative compounds of formula (1) include:

2-Piperazino-6-methyl-5-oxo-5,6-dihydro(7H)pyrro-[3,4-d]pyrimidine,

2-(4-Methylpiperazino-6-methyl-5-oxo-5,6-dihydro-(7H)pyrro[3,4 -d]pyrimidine,

2-(4-Ethylpiperazino-6-methyl-5-oxo-5,6-dihydro-(7H)pyrro[3,4-d]pyrimidine,

2-Piperidino-6-methyl-5-oxo-5,6-dihydro(7H)pyrro-[3,4-d]pyrimidine,

2-(4-Methylpiperidino)-6-methyl-5-oxo-5,6-dihydro(7H)pyrro[3,4-d]pyrimidine

2-(4-Ethylpiperidino)-6-methyl-5-oxo-5,6-dihydro-(7H)pyrro[3,4-d]pyrimidine

2-Morpholino-6-methyl-5-oxo-5,6-dihydro(7H)pyrro-[3,4-d]pyrimidine,

2-Thiomorpholino-6-methyl-5-oxo-5,6-dihydro(7H)-pyrro[3,4-d]pyrimidine,

2-Piperazino-6-ethyl-5-oxo-5,6-dihydro(7H)pyrro-[3,4-d]pyrimidine,

2-Piperazino-6-isopropyl-5-oxo-5,6-dihydro(7H)-pyrro[3,4-d]pyrimidine,

2-Piperazino-6-n-butyl-5-oxo-5,6-dihydro(7H)-pyrro[3,4-d]pyrimidine,

2-Piperazino-6-sec.-butyl-5-oxo-5,6-dihydro(7H)-pyrro[3,4-d]pyrimidine,

2-Piperazino-6-t-butyl-5-oxo-5,6-dihydro(7H)-pyrro[3,4-d]pyrimidine,

2-Piperazino-4,6-dimethyl-5-oxo-5,6-dihydro(7H)-pyrro[3,4-d]pyrimidine,

2-Piperazino-6,7-dimethyl-5-oxo-5,6-dihydro(7H)-pyrro[3,4-d]pyrimidine,

2-Piperazino-6,7,7-trimethyl-5-oxo-5,6-dihydro-(7H)pyrro[3,4-d]pyrimidine,

2-Piperidino-4,6-dimethyl-5-oxo-5,6-dihydro(7H)-pyrro[3,4-d]pyrimidine,

2-Piperidino-6,7,7-trimethyl-5-oxo-5,6-dihydro-(7H)pyrro[3,4-d]pyrimidine,

2-Piperazino-7-methyl-6-ethyl-5-oxo-5,6-dihydro-(7H)pyrro[3,4-d]pyrimidine, and

2-Piperazino-4-methyl-6-ethyl-5-oxo-5,6-dihydro-(7H) pyrro[3,4-d]pyrimidine.

Typical illustrative compounds of formula (2) include:

2-Piperazino-7-methyl-6-oxo-5,6-dihydro(7H)pyrro-[2,3-d]pyrimidine,

2-(4-Methylpiperazino)-7-methyl-6-oxo-5,6-dihydro(7H)pyrro[2,3-d]pyrimidine

2-(4-Ethylpiperazino)-7-methyl-6-oxo-5,6-dihydro-(7H)pyrro[2,3-d]pyrimidine

2-(4-N-Acetylpiperazino)-7-methyl-6-oxo-5,6-dihydro(7H)pyrro[2,3-d]pyrimidine,

2-Piperidino-7-methyl-6-oxo-5,6-dihydro(7H)pyrro-[2,3-d]pyrimidine,

2-(4-Methylpiperidino)-7-methyl-6-oxo-5,6-dihydro(7H)pyrro[2,3-d]pyrimidine

4-(Ethylpiperidino)-7-methyl-6-oxo-5,6-dihydro-(7H)pyrro[2,3-d]pyrimidine,

2-Morpholino-7-methyl-6-oxo-5,6-dihydro(7H)pyrro-[2,3-d]pyrimidine,

2-Thiomorpholino-7-methyl-6-oxo-5,6-dihydro(7H)-pyrro[2,3-d]pyrimidine,

2-Piperidino-7-ethyl-6-oxo-5,6-dihydro(7H)pyrro-[2,3-d]pyrimidine,

2-Piperidino-7-n-propyl-6-oxo-5,6-dihydro(7H)-pyrro[2,3-d]pyrimidine,

2-Piperidino-7-isopropyl-6-oxo-5,6-dihydro(7H)-pyrro[2,3-d]pyrimidine,

2-Piperidino-7-n-butyl-6-oxo-5,6-dihydro(7H)-pyrro[2,3-d]pyrimidine,

2-Piperidino-7-t-butyl-6-oxo-5,6-dihydro(7H)-pyrro[2,3-d]pyrimidine,

2-Piperidino-5-methyl-6-oxo-5,6-dihydro(7H)pyrro-[2,3-d]pyrimidine,

2-Piperazino-5-methyl-6-oxo-5,6-dihydro(7H)pyrro-[2,3-d]pyrimidine,

2-Piperazino-4,7-dimethyl-6-oxo-5,6-dihydro(7H)-pyrro[2,3-d]pyrimidine,

2-Piperidino-5,7-dimethyl-6-oxo-5,6-dihydro(7H)-pyrro[2,3-d]pyrimidine,

2-Piperidino-5,5,7-trimethyl-6-oxo-5,6-dihydro-(7H)pyrro[2,3-d]pyrimidine,

2-Piperazino-5,7-dimethyl-6-oxo-5,6-dihydro(7H)-pyrro[2,3-d]pyrimidine,

2-Piperazino-5,5,7-trimethyl-6-oxo-5,6-dihydro-(7H)pyrro[2,3-d]pyrimidine,

2-Piperidino-4-methyl-7-ethyl-6-oxo-5,6-dihydro-(7H)pyrro[2,3-d]pyrimidine, and

2-Piperidino-5-methyl-7-ethyl-6-oxo-5,6-dihydro-(7H)pyrro[2,3-d]pyrimidine.

The method embodiment is not specifically directed to treating a pathological condition, injury or disease state but rather the rejuvenation of a subject, and/or tissues of an subject.

In certain embodiments the pyrimidine derivative of formula (I) is MS-818, or 2-piperadino-6-methyl-5-oxo-5,6-dihydro(7H) pyrrolo[2,3-d]pyrimidine maleate (the C₄H₄O₄ maleate salt), as disclosed in U.S. Pat. No. 4,959,368, incorporated by reference herein. In certain in vivo embodiments, the pyrimidine derivatives of formulae (I) and (II) is administered at a concentration of between about 0.01 mg/kg/day to 50 mg/kg/day, more preferably between about 0.1 mg/kg/day to 10 mg/kg/day, even more preferably between about 1 mg/kg/day to 5 mg/kg/day, and even more preferably about 3 mg/kg/day. In these embodiments, the pyrimidine derivatives of formulae (I) and (II) is administered for between about 1 and 60 days, or more preferably between about 1 and 30 days, or more preferably between about 1 and 15 days, or even more preferably between about 1 and 10 days, or more preferably between about 2 and 7 days, or even more preferably about 5 days. In certain others of these embodiments, the methods further comprise the step of administering a growth factor. In certain embodiments, the growth factor comprises fibroblast growth factor, epidermal growth factor or a combination thereof.

### Increasing and/or Maintaining Vigor in a Subject

In one embodiment, the subject invention pertains to a method of increasing vigor in a subject comprising administering a therapeutically effective amount of a composition comprising a SCPA. In a specific embodiment, increasing vigor in a subject comprises oral administration of the composition. In another embodiment, the subject invention pertains to a method of increasing vigor of one or more tissues in a subject comprising administering a therapeutically effective amount of a composition comprising a SCPA to said subject in such manner as to bring said SCPA in contact with a target stem cell population. As used herein, the term ‘vigor’ when used in the context of a subject's overall disposition without reference to administration to a tissue refers to the level of physical strength, stamina, energy, and/or mental strength of the subject; when used in the context of particular tissue(s) it refers to the level of juvenescence and robustness of the target tissue of the subject. Thus, an increase in vigor caused by administration of an SCPA may pertain to an increase in one or more of a subject's disposition characteristics or to specific tissue characteristics as compared to that where no administration of an SCPA is made. It is contemplated that the increase in vigor is not necessarily targeted to a pathology or disease. Accordingly, the methods taught herein can serve as a vigor boost to even a clinically healthy subject.

In a further embodiment, the subject invention pertains to maintaining juvenescence of tissues in a subject comprising administering a therapeutically effective amount of a composition comprising a SCPA.

The active compound of certain composition embodiments, SCPA, may be included in a pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo systems (see, e.g., Rosenthal et al. (1996) Antimicrob. Agents Chemother. 40(7):1600-1603; Dominguez et al. (1997) J. Med. Chem. 40:2726-2732; Clark et al. (1994) Molec. Biochem. Parasitol. 17:129; Ring et al. (1993) Proc. Natl. Acad. Sci. USA 90:3583-3587; Engel et al. (1998) J. Exp. Med. 188(4):725-734; Li et al. (1995) J. Med. Chem. 38:5031) and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art.

Typically, a therapeutically effective dosage should produce a serum concentration of active compound of from about 0.1 ng/ml to about 50-100 μg/ml. The pharmaceutical compositions typically should provide a dosage of from about 0.001 mg to about 2000 mg of compound per kilo-gram of body weight per day. Pharmaceutical dosage unit forms are prepared to provide from about 1 mg to about 1000 mg and preferably from about 10 to about 500 mg of the essential active ingredient or a combination of essential ingredients per dosage unit form.

The active compound may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Preferred pharmaceutically acceptable derivatives include acids, bases, enol ethers and esters, salts, esters, hydrates, solvates and prodrug forms. The derivative is selected such that its pharmacokinetic properties are superior to the corresponding neutral compound.

Thus, effective concentrations or amounts of one or more of the compounds described herein or pharmaceutically acceptable derivatives thereof are mixed with a suitable pharmaceutical carrier or vehicle for systemic, topical or local administration to form pharmaceutical compositions. The concentration of active compound in the composition will depend on absorption, inactivation, excretion rates of the active compound, the dosage schedule, amount administered, particular formulation as well as other factors known to those of skill in the art.

The compositions are intended to be administered by a suitable route depending on the target outcome, including orally, parenterally (including intraventrically), rectally, topically (including intraocularly) and locally. Parenteral administration, generally characterized by injection, either subcutaneously, intramuscularly or intravenously is also contemplated herein. Parenteral administration as contemplated herein also pertains to other modes of internal administration that do not involve oral administration, such as direct introduction into specific tissues and organs including, but not limited to, into the central nervous system (e.g. intraventrically), the liver, heart, pancreas, kidneys, bone, and/or connective tissue (including tendons, ligaments, fascia etc.). For oral administration, capsules and tablets are presently preferred. The compositions are in liquid, semi-liquid or solid form and are formulated in a manner suitable for each route of administration. In certain embodiments, the preferred modes of administration include parenteral and oral modes of administration.

In another embodiment, the subject invention pertains to a method of increasing density of bone in a subject comprising administering an effective amount of a composition comprising a SCPA according to a regimen such that new bone is formed and bone density is increased.

Solutions or suspensions used for parenteral, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Certain composition embodiments can contain along with the active compound: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polvinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound in an amount sufficient to alleviate the symptoms of the treated subject.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain 0.001%-100% active ingredient, preferably 0.1-85%, typically 75-95%.

The active compounds or pharmaceutically acceptable derivatives may be prepared with carriers that protect the compound against rapid elimination from the body, such as time release formulations or coatings.

Of interest herein are also lyophilized powders, which can be reconstituted for administration as solutions, emulsions and other mixtures. They may also be reconstituted and formulated as solids or gels. The sterile, lyophilized powder is prepared by dissolving a SCPA compound in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (10-1000 mg, preferably 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, preferably 5-35 mg, more preferably about 9-30 mg of lyophilized powder, is added per mL of sterile water or other suitable carrier. The precise amount depends upon the selected compound. Such amount can be empirically determined.

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

The compounds may be formulated for local or topical application, such as for topical application to the skin and mucous membranes, such as in the eye, in the form of gels, liquid drops, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

In a specific embodiment, the subject invention pertains to a method of increasing vigor of a subject's skin comprising topically administering a therapeutically effective amount of a composition comprising a SCPA according to a regimen by which said skin vigor is increased. Increased skin vigor may pertain to improved skin tone, diminishing of wrinkles, improved complexion, improved skin smoothness, improved, skin resiliency, flexibility and/or elasticity or other characteristics representing increased juvenescence or appearance thereof, of the skin of the subject. In another specific embodiment, the subject invention pertains to a method of maintaining vigor of a subject's skin comprising a comprising topically administering a therapeutically effective amount of a composition comprising a SCPA according to a regimen by which said skin vigor is maintained over a period of time. A regimen may pertain to a certain amount, applied according to a certain frequency over a certain period of time.

According to certain embodiments of the topical compositions of the present invention also includes at least one of the following: a surface smoother, a skin plumper, an optical diffuser, a sunscreen, an exfoliation promoter, and an antioxidant.

(i) A surface smoother provides the functional benefits of enhancing skin smoothness and reducing the appearance of fine lines and coarse wrinkles. Examples include silicas, talcs, isopropyl myristate, petrolatum, isopropyl lanolate, silicones (e.g., methicone, dimethicone), polymethylmethacrylate (PMMA), or any mixtures thereof. The surface smoother is preferably present from about 0.1 wt % to about 50 wt % of the total weight of the composition.

(ii) A skin plumper serves as a collagen enhancer to the skin. An example of a suitable, and preferred, skin plumper is palmitoyl oligopeptide. Other skin plumpers are collagen and/or glycosaminoglycan (GAG) enhancing agents. The skin plumper is preferably present from about 0.1 wt % to about 20 wt % of the total weight of the composition.

(iii) An optical diffuser is a particle that changes the surface optometrics of skin, resulting in a visual blurring and softening of, for example, lines and wrinkles. Examples of optical diffusers that can be used in the present invention include, but are not limited to, boron nitride, mica, nylon, polyurethane powder, sericite, silica, silicone powder, talc, Teflon, titanium dioxide, zinc oxide, or any mixtures thereof. The optical diffuser is preferably present from about 0.01 wt % to about 20 wt % of the total weight of the composition.

(iv) A sunscreen protects the skin from damaging ultraviolet rays. In an illustrative embodiment of the present invention, the sunscreen would provide both UVA and UVB protection, by using either a single sunscreen or a combination of sunscreens. Among the sunscreens that can be employed in the present compositions are avobenzone, cinnamic acid derivatives (such as octylmethoxy cinnamate), octyl salicylate, oxybenzone, titanium dioxide, zinc oxide, or any mixtures thereof. Preferably, the sunscreen is present from about 1 wt % to about 30 wt % of the total weight of the composition. In particular, the addition of a sunscreen is preferred to prevent/reduce the photodegradation of retinoid while in the package and/or on the skin.

Other routes of administration, such as transdermal patches and rectal administration are also contemplated herein. For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm.

Tablets and capsules for rectal administration are manufactured using the same pharmaceutically acceptable substance and by the same methods as for formulations for oral administration.

The SCPA compounds or pharmaceutically acceptable derivatives may be packaged as articles of manufacture containing packaging material, a compound or pharmaceutically acceptable derivative thereof provided herein.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,352. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated for treatment and prevention of insulin resistance.

### Enhancement of Other Therapies

In certain embodiments, SCPA compounds as described herein may be used in conjunction with other therapies to enhance their effect. Of particular interest are therapies that involved the transplantation or implantation of tissues or cells. For example, SCPA compounds may be administered in conjunction with a bone marrow transplant and/or used to treat the bone marrow prior to transplantation. Administration of the SCPA enhances proliferation of the stem cells in the bone marrow, which provides for an improved therapeutic outcome, including a more abundant source of stem cells and quicker production of blood in the bone marrow recipient.

### Co-Administration of Stem Cells With a SCPA

In another embodiment, the subject invention pertains to a method of improving the outcome of a stem cell implantation therapy comprising the co-administration of stem cells with a SCPA. The cells are administered by injecting one or a plurality of stem cells with a syringe, inserting the stem cells with a catheter or surgically implanting the stem cells. In certain embodiments, the stem cells are administered into a body cavity fluidly connected to a target tissue. In certain preferred embodiments, the body cavity is a brain ventricle. In other embodiments, the stem cells are inserted using a syringe or catheter, or surgically implanted directly at the target tissue site. In other embodiments, the stem cells are administered systemically (e.g., parenterally).

A number of different stem cells are appropriate for increasing vigor as taught herein. Examples include, but are not limited to, mesenchymal stem cells (MeSCs), neural stem cells (NSCs), hematopoietic stem cells (HSCs). U.S. application Set. Nos. 11/258,401; 11/258,603; 11/258,392 and 11/258,360 discuss various methods for biasing potency and/or differentiation of stem cells, and are incorporated herein by reference. Stem cells may be purchased from commercially available sources, see Neuroreport, Vol 12 No 6 8 May 2001 and Restorative Neurology and Neuroscience 22 (2004) 459-468, or procured from autogenic, allogenic or xenogenic sources according to known techniques, see for example U.S. Patent Publication 20060078993; Br. J. Haematol. (2000) 109, 235-242; and International Patent Application WO 03/070922. Population of stem cells can be derived from multiple sources, including but not limited to, brain-derived neural stem cells, bone marrow derived mesenchymal stem cells, adipose-derived mesenchymal stem cells, blood-derived hematopoietic stem cells, cord-blood-derived stem cells. Cells could be derived from embryonic stem cells following treatment to induce differentiation toward a specific cell lineage.

In view of the teachings herein, one skilled in the art will appreciate that cells may be administered to an animal by a number of methods, including, but not limited direct injection into a target tissue or distal injection from which cells are transported or migrate to a target tissue.

In another embodiment, cells are administered through parenteral injection. In an optimum embodiment, hematopoietic stem cells are injected into the blood. The inventors believe that the hematopoietic stem cells will travel into the blood and broadly revitalize tissues in the subject's body, and that co-administration would enhance this effect. In particular, the inventors believe that stamina and energy would be increased via this embodiment.

In one embodiment where an autologous cell sample is produced, hematopoietic stem cells are procured from the bone marrow of a subject. These procured cells are cultured and expanded to produce an expanded sample of cells. The expanded sample is administered to the bloodstream, muscle tissue, connective tissue, and/or organ of the horse from which they were procured. In an alternative embodiment, an allogenic (same species different subject) cell sample is produced whereby cells from one subject are procured and processed and then administered to a different subject.

In a specific example, adult somatic cells including but not limited to white blood cells, fibroblasts, mesenchymal stem cells, and skin cells can be treated with nucleotide derivatives such as BrdU or 5-azacytidine to epigenetically modify the cells to increase their developmental potential. Additionally, cells can be treated with genes that expand the potency of cells including but not limited to genes that are responsible for maintaining the properties of embryonic stem cells such as nanog.

Cells can be positively selected for using cell-specific markers including but not limited to CD34, CD133 (hematopoietic stem cells), STRO-1, SH2, SH3, (mesenchymal stem cells), nestin, PSA-NCAM (neural stem cells). Cells can also be purified through negative selection by selecting out cells that express markers not present in the desired cell population. For example, lineage markers indicating differentiation such as CD38, CD45, and “Lin” markers (blood cell lineage proteins expressed in differentiating blood cells) can select out white blood cells from a mixture of cells.

Cells can also be selected using physical properties such as growth characteristics, adhesion, and/or density. For example, a density gradient can separate red blood cells from a solution of bone marrow and adhesion of cells to a culture flask can select for mesenchymal cells (while hematopoietic cells remain non-adherent).

As discussed above, stem cells may be procured using convention techniques in the stem cell art. In one example, stem cells are obtained from bone marrow or blood. See, for example, Friedenstein A J, Gorskaja J F, Kulagina N N, Exp Hematol. 1976 September; 4(5):267-74; and Caplan A I J, Orthop Res. 1991 September; 9(5):641-50. A bone marrow sample is explanted from a donor and hematopoietic stem cells are isolated from the marrow sample according to known techniques, including use flow cytometry or an affinity column. See, for example, U.S. Patent Publication Nos. 20040265996; 20050158857; 20060088890; and 20060073124. In a specific embodiment, hematopoietic cells are isolated using positive or negative selection. See U.S. Patent Publication No. 20060073124. Negative selection removes unwanted cells using certain markers such as C45 or positive selection using CD34.

Once cells are isolated, they may be cultured, expanded, subjected to external biasing factors and/or genetically modified by introduction of genes encoding for biasing factors, see U.S. application Ser. Nos. 11/258,401; 11/258,603; 11/258,392 and 11/258,360 are incorporated herein by reference. Mesenchymal cells may be isolated by similar techniques or through the use of a gradient, such as FICOL gradient. Mesenchymal cells in a bone marrow sample will attach to surface, whereas other undesired cells will not attach and remain in the media. The media with the undesired cells is removed leaving the desired mesenchymal cells. The mesenchymal cells, as with other cells, are cultured, expanded, subjected to external biasing factors and/or genetically modified.

As noted above, cells can be harvested from a subject and autologously transplanted back following treatment and/or expansion of cells. However, it may be more efficient to obtain stem cells from readily accessible source. For example, according to another embodiment, cells are harvested, catalogued according to predetermined characteristics, e.g., genotyping, blood type, major histocompatibility complex, or genomic characteristics, and stored under appropriate conditions (typically by freezing) to keep the stem cells alive and functioning.

The inventors have found that co-administration of secondary cells with primary cells can influencing the transplant loci so as to be more conducible to acceptance and differentiation of the primary cells to their target cell type. For example, mesenchymal cells may reduce the amount of inflammation at the site of implantation of hematopoietic or neural stem cells.

In a further embodiment, stem cells are co-administered with a nutrient composition. The nutrient composition may be administered prior to, concurrent to, or subsequent to the administration of the stem cells. The nutrient composition may be administered in a similar mode as the stem cells or by a separate mode. For example, stem cells may be administered by parenteral injection while the nutrient composition is administered by oral ingestion.

In another embodiment, external factors are co-administered with the stem cells. Typically, such factors are provided at the site of administration. Such external factors may optimize the implantation site environment, or may serve to bias differentiation of the implanted stem cells.

In a further embodiment, the subject invention pertains to a method of increasing the longevity of a healthy subject comprising administering a longevity enhancing composition to said subject, said longevity-enhancing composition comprising a SCPA. Optionally, the longevity-enhancing composition is co-administered autologous stem cells or exogenous stem cells, or both, as discussed above, optionally along with a pharmaceutically acceptable carrier. It is important to note that the longevity enhancing composition is not targeted to treating a pathology in said subject.

### Psychiatric Applications

Recent evidence suggests that certain psychiatric disorders may be caused by a modulation of neurogenesis in certain regions of the brain. For example, it has been shown that depression appears to be related to a decrease or down-modulation of neurogenesis in the hippocampus. Dranovsky and Hen, Hippocampal Neurogenesis: Regulation by Stress and Anti-depressants, Biol Psychiatry 2006 59:1136-1143; Malberg, Adult Hippocampal Neurogenesis and Depression, J. Psychiatry Neurocsi 2004 29(3):196-205. Accordingly, given the stem proliferative properties of the compounds taught herein, it is believed that such compounds may be utilized to treat psychiatric disorders that have been shown to be related to regulation regulation of neurogenesis in the brain. According to one embodiment, the subject invention pertains to a method of treating and/or preventing a neurogenesis related psychiatric disorder comprising administering a therapeutically effective amount of a SCPA. This may achieved by an oral or parenteral administration generally including any suitable systemic administration. In a certain embodiment, the SCPA is administered by inhalation, intraocular, oral, intramuscular, intravenous, transdermal or local to the affected brain region.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

The teachings of all references cited herein are incorporated by reference in their entirety to the extent not inconsistent with the teachings herein.

What is claimed is:

1. A method of improving skin tone of a subject's skin comprising topically administering to non-wounded skin of said subject a therapeutically effective amount of a composition comprising 2-Piperadino-6-methyl-5-oxo-5,6-dihydro(7H)pyrro-[3,4-d]pyrimidine, or pharmaceutically acceptable salt thereof.

2. The method of claim 1, wherein said composition further comprises a surface smoother, a skin plumper, an optical diffuser, a sunscreen, an exfoliation promoter, or an antioxidant, or a combination thereof.

3. The method of claim 1, wherein the composition comprises 2-Piperadino-6-methyl-5-oxo-5,6-dihydro(7H)pyrro-[3,4-d]pyrimidine maleate [chemical structure]
